(19) United States
(12) Patent Application Publication
(10) Pub. No.: US 2023/0181601 A1
(43) Pub. Date: Jun. 15, 2023

(54) TREATMENT OF NON-ALCOHOLIC STEATOHEPATITIS (NASH)

(71) Applicant: DURECT CORPORATION, Cupertino, CA (US)

(72) Inventor: WEIQI LIN, Emerald Hills, CA (US)

(21) Appl. No.: 17/924,101

(22) PCT Filed: May 21, 2021
(86) PCT No.: PCT/US21/33743

§ 371 Date: Nov. 8, 2022

Related U.S. Application Data
(60) Provisional application No. 63/029,361, filed on May 22, 2020.
(60) Provisional application No. 63/030,207, filed on May 26, 2020.
(60) Provisional application No. 63/113,116, filed on Nov. 12, 2020.
(60) Provisional application No. 63/146,555, filed on Feb. 5, 2021.

(51) Int. Cl. A61K 31/575; A61K 9/00; A61K 45/06; A61P 1/16

(52) U.S. Cl. CPC A61K 31/575; A61K 9/0053; A61K 45/06; A61P 1/16

(57) ABSTRACT

Methods of treating non-alcoholic steatohepatitis (NASH) are provided. For instance, the methods comprise administering 5-cholesten-3,25-diol, 3-sulfate (25HC3S) ora salt thereof.

### CROSS-REFERENCE TO RELATED APPLICATION

This application claims the benefit of priority to U.S. Provisional Patent Application Ser. No. 63/029,361 filed on May 22, 2020; U.S. Provisional Patent Application Ser. No. 63/030,207 filed on May 26, 2020; U.S. Provisional Patent Application Ser. No. 63/113,116 filed on Nov. 12, 2020; and U.S. Provisional Patent Application Ser. No. 63/146,555 filed on Feb. 5, 2021, the disclosures of which applications are herein incorporated by reference.

### BACKGROUND

Non-alcoholic steatohepatitis (NASH) is an extreme and progressive form of non-alcoholic fatty liver disease (NAFLD) that is not linked to alcohol consumption and is further accompanied by inflammation (hepatitis). NASH is accompanied by ballooning degeneration of hepatocytes (also referred to herein as “hepatocyte ballooning”), which refers to the increase in size (i.e., ballooning) of cells during this process that is considered to be a form of apoptosis. Ballooned cells are typically two to three times the size of adjacent hepatocytes and characterized by a wispy cleared cytoplasm on H&E stained sections. Liver cell death and the inflammatory response lead to activation of stellate cells, which play a pivotal role in hepatic fibrosis. Further disease progression leads to cirrhosis and hepatocellular carcinoma (HCC), resulting in liver failure and, ultimately, death.

For patients suffering from early stages of NASH, lifestyle intervention, such as significant weight reduction, may slow or even reverse the process of steatosis. However, for patients with advanced NASH, there are no currently available therapies. Given the severity of fatty liver disease (FLD) and NASH and unmet clinical need, an effective therapeutic treatment is urgently needed.

U.S. Pat. No. 8,399,441, which is incorporated by reference herein, discloses the use of 5-cholesten-3,25-diol, 3-sulfate (25HC3S) and salts thereof for the treatment of conditions associated with high cholesterol and/or high triglycerides and/or inflammation (e.g., hypercholesterolemia, hypertriglyceridemia, non-alcoholic fatty liver diseases (e.g., NASH), atherosclerosis, etc.).

U.S. Pat. No. 9,034,859, which is incorporated by reference herein, discloses the use of 25HC3S and salts thereof for prevention and treatment of liver damage or disease (e.g., NASH).

KEMP et al., “Safety and pharmacokinetics of DUR-928 in patients with non-alcoholic steatohepatitis—A Phase 1b study,” Poster session presented at The International Liver Congress (2017), discloses a Phase 1b single dose ranging (50 mg and 200 mg) safety/PK study of orally-administered DUR-928 in biopsy-confirmed NASH patients and matched control subjects (MCS).

SHAH et al., “Pharmacokinetic and Pharmacodynamic Response in Individual NASH Patients Receiving Two Dose Levels of DUR-928,” NASH Summit—2019 (April 22-25, 2019) discloses oral administration of 5-cholesten-3ρ,25-diol 3-sulfate (25HC3S) to NASH patients. The patients received both 50 mg and 200 mg doses administered approximately two months apart. SHAH et al. concludes that there were no dose-dependent changes of biological responses between 50 mg and 200 mg dose levels.

There is an urgent need for improved methods to treat NASH.

### SUMMARY

The present disclosure provides a variety of methods of treating non-alcoholic steatohepatitis (NASH). The methods involve administering an effective amount of 5-cholesten-3,25-diol, 3-sulfate (25HC3S) or salt thereof. In certain instances, the methods involve administering 25HC3S or salt thereof in an amount ranging from 1 mg/day to 100 mg/day.

The results of the present disclosure are surprising. The results are surprising at least because the recited dose resulted in reduced liver fat (e.g., as measured by MRI-PDFF) compared with higher doses. It has now been unexpectedly found that the specific dosage regimens of the present invention may lead to improved clinical outcomes, as for instance further discussed and evidenced herein.

Further aspects of the disclosure:
1. A method of treating non-alcoholic steatohepatitis (NASH) in a human subject in need thereof, the method comprising orally administering to the subject 5-cholesten-3,25-diol, 3-sulfate (25HC3S) or salt thereof in an amount ranging from 1 mg/day to 100 mg/day.
2. A method of lowering serum alanine aminotransferase (ALT) levels in a human subject having non-alcoholic steatohepatitis (NASH), comprising:
   orally administering to the subject 5-cholesten-3,25-diol, 3-sulfate (25HC3S) or salt thereof in an amount ranging from 1 mg/day to 100 mg/day.
3. A method of lowering liver stiffness in a human subject having non-alcoholic steatohepatitis (NASH), comprising:
   orally administering to the subject 5-cholesten-3,25-diol, 3-sulfate (25HC3S) or salt thereof in an amount ranging from 1 mg/day to 100 mg/day.

4. A method of lowering serum triglycerides in a human subject having non-alcoholic steatohepatitis (NASH), comprising:

orally administering to the subject 5-cholesten-3,25-diol, 3-sulfate (25HC3S) or salt thereof in an amount ranging from 1 mg/day to 100 mg/day.

5. A method of lowering serum triglycerides in a human subject having non-alcoholic steatohepatitis (NASH) and having triglycerides ≥200 mg/dL prior to treatment, comprising:
orally administering to the subject 5-cholesten-3,25-diol, 3-sulfate (25HC3S) or salt thereof in an amount ranging from 1 mg/day to 100 mg/day.
6. The method according to any one of aspects 1 to 5, wherein the orally administering comprises orally administering the 25HC3S or salt thereof ranging from about 10 mg/day to about 80 mg/day.
7. The method according to any one of aspects 1 to 5, wherein the orally administering comprises orally administering the 25HC3S or salt thereof ranging from about 30 mg/day to about 70 mg/day.
8. The method of any one of aspects 1 to 7, wherein a total amount per kg of 25HC3S or salt thereof that is orally administered to the subject ranges from about 0.1 mg/kg/day to about 5 mg/kg/day.
9. The method of aspect 8, wherein the total amount per kg ranges from about 0.2 mg/kg/day to about 4 mg/kg/day.
10. The method of aspect 8, wherein the total amount per kg ranges from about 0.3 mg/kg/day to about 3 mg/kg/day.
11. The method of aspect 8, wherein the total amount per kg ranges from about 0.4 mg/kg/day to about 2 mg/kg/day.
12. The method of any one of aspects 1 to 11, wherein the orally administering comprises orally administering a plurality of doses of the 25HC3S or salt thereof
13. The method of aspect 12, wherein the doses are orally administered at a frequency ranging from once weekly to three times a day.
14. The method of aspect 13, wherein the doses are orally administered once a day.
15. The method of aspect 13, wherein the doses are orally administered twice a day.
16. The method of any one of aspects 12 to 15, wherein the orally administering comprises orally administering for a dosing period of at least 7 days, such as at least 14 days, at least 28 days, at least 3 months, at least 6 months, or at least 1 year.
17. The method of any one of aspects 1 to 16, wherein the 25HC3S or salt thereof is orally administered in a formulation comprising the 25HC3S or salt thereof and a pharmaceutically acceptable carrier.
18. The method of any one of aspects 1 to 17, wherein the 25HC3S or salt thereof comprises a salt of 25HC3S.
19. The method of aspect 18, wherein the salt of 25HC3S is sodium salt.
20. The method of any one of aspects 1 to 19, wherein the human subject has a magnetic resonance imaging-proton density fat fraction (MRI-PDFF) prior to treatment of at least 5%.
21. The method of any one of aspects 1 to 20, wherein the human subject has a magnetic resonance elastography (MRE) prior to treatment ≥2.75 kPa.
22. The method of any one of aspects 1 to 21, wherein the subject exhibits a half-life time of 25HC3S in the plasma after administration (T_{1/2}) ranging from about 1 hour to about 5 hours or from about 1.5 hour to about 4 hours.
23. The method of any one of aspects 1 to 22, wherein the subject exhibits a Cmax of 25HC3S ranging from about 25 ng/mL to about 200 ng/mL, from about 50 ng/mL to about 150 ng/mL, or from about 75 ng/mL to about 125 ng/mL.
24. The method of any one of aspects 1 to 23, wherein the subject exhibits a Cmax of 25HC3S ranging from about 100 ng/mL to about 300 ng/mL, from about 120 ng/mL to about 250 ng/mL, or from about 150 ng/mL to about 200 ng/mL, per 100 mg of orally administered 25HC3S or salt thereof.
25. The method of any one of aspects 1 to 24, wherein the subject exhibits an AUCinf of 25HC3S ranging from about 300 ng*h/mL to about 1000 ng*h/mL, about 400 ng*h/mL to about 900 ng*h/mL, or from about 500 ng*h/mL to about 800 ng*h/mL.
26. The method of any one of aspects 1 to 25, wherein the subject exhibits an AUCinf of 25HC3S ranging from about 600 ng*h/mL to about 1000 ng*h/mL, about 700 ng*h/mL to about 900 ng*h/mL, or from about 800 ng*h/mL to about 900 ng*h/mL, per 100 mg of orally administered 25HC3S or salt thereof
27. The method of any one of aspects 1 to 29, wherein the subject exhibits an apparent volume of distribution (Vz/F) of 25HC3S ranging from about 300 L to about 1000 L, about 400 L to about 900 L, or from about 500 L to about 800 L.
28. The method of any one of aspects 1 to 27, wherein the subject exhibits an apparent clearance (CL/F) of 25HC3S ranging from about 100 L to about 200 L/h, about 110 L/h to about 180 L/h, or from about 120 L/h to about 160 L/h.
29. The method of any one of aspects 1 to 28, wherein the subject is taking a lipid lowering drug, such as at least one of a statin, fenofibrate, omega-3 fatty acid, icosapent ethyl, and fish oil, or further comprising administering a lipid lowering drug, such as at least one of a statin, fenofibrate, omega-3 fatty acid, icosapent ethyl, and fish oil, to the subject.
30. The method of any one of aspects 1 to 29, wherein the subject is taking at least one of atorvastatin, fluvastatin, lovastatin, pitavastatin, pravastatin, rosuvastatin, and simvastatin, or further comprising administering to the subject at least one of atorvastatin, fluvastatin, lovastatin, pitavastatin, pravastatin, rosuvastatin, and simvastatin.
31. 5-cholesten-3,25-diol, 3-sulfate (25HC3S) or salt thereof for use in a method of treating non-alcoholic steatohepatitis (NASH) in a human subject in need thereof, wherein the method is as defined in any one of aspects 1 to 30.
32. 5-cholesten-3,25-diol, 3-sulfate (25HC3S) or salt thereof for use in a method of treating non-alcoholic steatohepatitis (NASH) in a human subject in need thereof, the human subject having triglycerides ≥200 mg/dL prior to treatment, wherein the method is as defined in any one of aspects 1 to 30.
33. Use of 5-cholesten-3,25-diol, 3-sulfate (25HC3S) or salt thereof in a method for the manufacture of a medicament for use in a method of treating non-alcoholic steatohepatitis (NASH) in a human subject in need thereof, wherein the method is as defined in any one of aspects 1 to 30.
34. Use of 5-cholesten-3,25-diol, 3-sulfate (25HC3S) or salt thereof in a method for the manufacture of a medicament for use in a method of treating non-alcoholic steatohepatitis (NASH) in a human subject in need thereof, the human subject having triglycerides ≥200 mg/dL prior to treatment, wherein the method is as defined in any one of aspects 1 to 30.
35. 5-cholesten-3,25-diol, 3-sulfate (25HC3S) or salt thereof for use in a method of treating non-alcoholic steatohepatitis (NASH) in a human subject in need thereof, wherein said human subject is receiving statin therapy.
36. 5-cholesten-3,25-diol, 3-sulfate (25HC3S) or salt thereof for use according to aspect 35, wherein said statin therapy comprises administration of at least one of atorvastatin, fluvastatin, lovastatin, pitavastatin, pravastatin, rosuvastatin, and simvastatin.
37. 5-cholesten-3,25-diol, 3-sulfate (25HC3S) or salt thereof for use according to aspect 35 or 36, wherein said method is a method as defined in any one of aspects 1-30, and optionally wherein the human subject has triglycerides ≥200 mg/dL prior to treatment.
38. 5-cholesten-3,25-diol, 3-sulfate (25HC3S) or salt thereof for use in a method of treating non-alcoholic steatohepatitis (NASH) in a human subject in need thereof by co-administration with at least one statin, optionally wherein said at least one statin comprises at least one of atorvastatin, fluvastatin, lovastatin, pitavastatin, pravastatin, rosuvastatin, and simvastatin.
39. 5-cholesten-3,25-diol, 3-sulfate (25HC3S) or salt thereof for use according to aspect 38, wherein said human subject is one receiving statin therapy prior to commencing said method, and optionally wherein said statin therapy comprises administration of the same statin or statins that is or are co-administered with said 25HC3S or salt thereof in said method.
40. 5-cholesten-3,25-diol, 3-sulfate (25HC3S) or salt thereof for use according to aspect 38 or 39, wherein said method is a method as defined in any one of aspects 1-30, and optionally wherein the human subject has triglycerides ≥200 mg/dL prior to treatment.
41. Use of 5-cholesten-3,25-diol, 3-sulfate (25HC3S) or salt thereof in a method for the manufacture of a medicament for use in a method of treating non-alcoholic steatohepatitis (NASH) in a human subject in need thereof, wherein said human subject is receiving statin therapy.
42. Use according to aspect 41, wherein said statin therapy comprises administration of at least one of atorvastatin, fluvastatin, lovastatin, pitavastatin, pravastatin, rosuvastatin, and simvastatin.
43. Use according to aspect 41 or 42, wherein said method of treating is a method as defined in any one of aspects 1-30, and optionally wherein the human subject has triglycerides ≥200 mg/dL prior to treatment.
44. Use of 5-cholesten-3,25-diol, 3-sulfate (25HC3S) or salt thereof in a method for the manufacture of a medicament for use in a method of treating non-alcoholic steatohepatitis (NASH) in a human subject in need thereof by co-administration with at least one statin, optionally wherein said at least one statin comprises at least one of atorvastatin, fluvastatin, lovastatin, pitavastatin, pravastatin, rosuvastatin, and simvastatin.
45. Use according to aspect 44, wherein said human subject is one receiving statin therapy prior to commencing said method, and optionally wherein said statin therapy comprises administration of the same statin or statins that is or are co-administered with said 25HC3S or salt thereof in said method.
46. Use according to aspect 44 or 45, wherein said method of treating is a method as defined in any one of aspects 1-30, and optionally wherein the human subject has triglycerides ≥200 mg/dL prior to treatment.

### BRIEF DESCRIPTION OF THE FIGURES

FIG. 1 depicts the mean pharmacokinetic (PK) parameters of subjects administered 25HC3S according to certain embodiments.

FIG. 2 depicts the pharmacokinetic (PK) plasma concentrations of 25HC3S for healthy and NASH subjects following administration of 50 mg 25HC3S according to certain embodiments.

FIG. 3 depicts the pharmacokinetic (PK) plasma concentrations of 25HC3S for healthy and NASH subjects following administration of 200 mg 25HC3S according to certain embodiments.

### DETAILED DESCRIPTION OF THE INVENTION

Methods for treating non-alcoholic steatohepatitis (NASH) are described herein. The methods include contacting the liver with 25HC3S or salt thereof. The contact generally involves administering to a human patient 25HC3S or salt thereof in an amount ranging from 1 mg/day to 100 mg/day.

As discussed above, the results of the present disclosure are surprising. The results are surprising at least because the recited dose resulted in reduced liver fat (e.g., as measured by MRI-PDFF) compared with higher doses.

### Definitions

The following definitions are used throughout:

“Treat” (treatment, treating, etc.) as used herein refers to administering 25HC3S or salt thereof to a human subject that: (1) already exhibits at least one symptom of NASH; and/or (2) is diagnosed as having NASH, such as by a trained clinical professional; and/or (3) is determined to have NASH based on laboratory (e.g., molecular indicators) or clinical tests of one or more body fluids, such as blood. In certain embodiments, subjects are diagnosed as having NASH by liver tissue biopsy. In other words, at least one parameter that is known to be associated with NASH has been measured, detected or observed in the subject. “Treatment” of NASH involves the lessening or attenuation, or in some instances, the complete eradication, of at least one symptom of NASH that was present prior to or at the time of administration of 25HC3S or salt thereof. In some embodiments, treating NASH according to the present disclosure is sufficient to improve laboratory or clinical indicators of NASH in the subject as described in greater detail below. In certain instances, the improvement in the laboratory or clinical indicators of NASH in the subject is such that the subject is considered to no longer have NASH.

“Liver dysfunction” denotes a condition or a state of health where the liver does not perform its expected function, such as where certain biological or molecular indicators are measured to be outside of normal physiologic ranges. Liver function represents the expected function of the liver within physiologic ranges. The person skilled in the art is aware of the respective function of the liver during medical examination. Liver dysfunction typically involves a clinical syndrome in which the development of progressive and potentially reversible physiological dysfunction in the liver, optionally in the absence of anatomic injuries.

“Liver failure” denotes liver dysfunction to such a degree that normal homeostasis cannot be maintained without external clinical intervention.

“CK-18” refers to cytokeratin-18 fragment, which has been identified as a noninvasive biomarker for NASH in that it is markedly increased in patients with NASH as determined by histology and higher blood plasma levels of the fragment correlate with the odds of having fibrosis on liver biopsy. See Feldstein et al., Hepatology, 50:1072-1078 (2009), incorporated by reference herein.

“Pharmaceutically acceptable” refers to a substance that does not interfere with the effectiveness of the biological activity of the active ingredient and is not toxic to the host to which it is administered.

### Methods of Treating Nash

The present disclosure provides a therapy for the treatment of NASH that comprises administering to a patient in need of treatment 25HC3S or salt thereof in an amount ranging from 1 mg/day to 100 mg/day. In some embodiments, the patient in need of treatment is a patient who has been diagnosed with NASH. In some embodiments, treatment with 25HC3S or salt thereof as described herein slows, stops, or improves NASH.

### Patient Population

Patients likely to benefit from the therapies of the present disclosure can be readily identified by a variety of means discussed herein or known to those of skill in the art. In addition, methods for determining whether a patient is responding to this therapy are also provided. In some embodiments, abdominal imaging tests, including ultrasound examination, computerized tomography (CT), and/or magnetic resonance imaging (MRI) can be used to diagnose patients with the disease, e.g., evaluate whether the disease is present and its severity. Such a non-invasive diagnosis can be more definitively confirmed by liver biopsy, if desired. In some embodiments, one or more biomarkers is used to diagnose NASH. In some embodiments, a patient to be treated in accordance with the present disclosure has received a primary diagnosis of NASH and is not being treated with 25HC3S or salt thereof for any other condition for which it is currently in clinical development (e.g., alcoholic hepatitis (AH) or COVID-19).

In some cases, the patient to be treated has a magnetic resonance imaging-proton density fat fraction (MRI-PDFF) >5%, such as >10%, >15%, >20%, >25%, or >30%. In some cases, the patient to be treated has an MRI-PDFF ranging from 4% to 60%, such as 5% to 50%, 10% to 40%, or 15% to 30%.

In some cases, the patient to be treated has a magnetic resonance elastography (MRE) ≥2 kPa, such as ≥2.5 kPa, ≥3 kPa, ≥3.5 kPa, ≥4.0 kPa, or ≥4.5 kPa. In some cases, the patient to be treated has an MRE ranging from about 2 kPa to about 10 kPa, such as about 3 kPa to about 8 kPa, or about 3.5 kPa to about 6 kPa.

In some cases, the patient to be treated has a Fibroscan® value ≥5 kPa, such as ≥7 kPa, ≥7.5 kPa, ≥12.5 kPa, or ≥14 kPa. In some cases, the patient to be treated has a Fibroscan® value ranging from about 7 kPa to about 75 kPa, such as about 7.5 kPa to about 60 kPa, about 8 kPa to about 50 kPa, or 10 kPa to about 40 kPa.

In some cases, the patient to be treated has a CAP score >200 dB/m, such as >250 dB/m, or >300 dB/m. In some cases, the patient to be treated has a CAP score ranging from about 200 dB/m to about 400 dB/m, such as about 250 dB/m to about 300 dB/m.

In some embodiments, the patient exhibits abnormal liver function, e.g., as determined by the presence of elevated serum aspartate aminotransferase (ALT), gamma glutamyl transpeptidase (GGT), total bilirubin (TBL), and/or alkaline phosphatase (ALP) levels. In some embodiments, a patient to be treated has an elevated ALT level, an elevated gamma glutamyl transpeptidase, and/or an elevated alkaline phosphatase level (e.g., a level that is about 1.5- to 4-fold above the upper limit of normal). In some cases, the patient to be treated has an ALT concentration >1 and <5 times upper limit of normal (ULN). In some cases, the patient to be treated has an ALT concentration <30 U/L, such as <20 UI. In some cases, the patient to be treated has an AST concentration <5× upper limit of normal (ULN). In some cases, the patient to be treated has GGT >15 U/L, such as GGT >30 U/L. In some cases, the patient to be treated has GGT ranging from 5 U/L to 500 U/L, such as 15 U/L to 400 U/L, 20 U/L to 350 U/L, or 30 U/L to 300 U/L. In some embodiments, a patient to be treated has an ALT level, gamma glutamyl transpeptidase level, and/or alkaline phosphatase level that is within the upper limit of normal.

In some cases, the patient to be treated has elevated lipid levels, especially elevated levels of serum triglycerides. In some cases, the patient has elevated serum cholesterol, including low density lipoprotein cholesterol (LDL-C) and triglycerides (TG). In some cases, the patient to be treated has low levels of HDL cholesterol. In some cases, the patient to be treated has hypertension. In some cases, the patient to be treated has cardiovascular disease. In some cases, the patient to be treated has chronic obstructive pulmonary disease (COPD). In some cases, the patient to be treated has chronic kidney disease (CKD). In some cases, the patient to be treated has diabetes.

In some embodiments, NASH is diagnosed using an imaging test. In some embodiments, NASH is diagnosed using a scoring system such as but not limited to fatty liver index, NAFLD liver fat score, NAFLD activity score, or hepatic steatosis index. In some embodiments, NASH is diagnosed using a NAFLD activity score (NAS), which provides a composite score based on the degree of steatosis (0-3), lobular inflammation (0-3), and hepatocyte ballooning (0-2). See Kleiner et al., Hepatology, 41:1313-1321 (2005); and Bugianesi et al., J Hepatology, 65:643-644 (2016). In some cases, the patient to be treated has stage 1, 2, or 3 fibrosis and a NAS≥4, with at least 1 point for each of steatosis, hepatocellular ballooning, and lobular inflammation.

NASH has been classified pathologically into type 1 and type 2 forms, of which the type 1 form is more commonly found in adult patients, while the type 2 form is more commonly found in children. Type 1 NASH is typically characterized by steatosis, hepatocyte ballooning, and perisinusoidal fibrosis. Type 2 NASH is typically characterized by steatosis, portal inflammation, and portal fibrosis. See, e.g., Schwimmer et al., Hepatology, 42:641-649 (2005). Further progression of NASH can lead to severe fibrosis, cirrhosis, and end-stage liver disease. In some embodiments, a patient to be treated has Type 1 NASH. In some embodiments, a patient to be treated has Type 2 NASH. In some embodiments, a patient to be treated has early-stage NASH. In some embodiments, a patient to be treated has middle-stage NASH. In some embodiments, a patient to be treated has late-stage NASH (e.g., has severe fibrosis and/or cirrhosis of the liver).

In some embodiments, NASH is diagnosed using an imaging test. In some embodiments, NASH is diagnosed using a scoring system such as but not limited to NAFLD activity score (e.g., a score of >5) or a steatosis, activity, and fibrosis (SAF) score, or a NAFLD fibrosis score; a serum biomarker (e.g., cytokeratin-18); or a combination thereof. See Bedossa et al., Hepatology, 56:1751-1759 (2012); Arab et al., Gastroenterol Hepatol, 40:388-394 (2017). In some embodiments, fibrosis is detected and/or measured using elastography (e.g., Fibroscan®).

In some embodiments, a patient to be treated is identified by use of one or more biomarkers such as CK-18. CK-18 levels, whether measured by immunohistochemistry, histology from liver biopsies, or via measurement of plasma levels in patients or individuals suspected of being at risk for the disease, will typically be elevated, relative to the levels measured in healthy individuals, in subjects in need of treatment. While the present disclosure is not to be limited to a particular or any proposed mechanism of action, decreased CK-18 levels in NASH patients would be expected to correlate with decreased liver cell apoptosis. In some embodiments, patients with NASH who are treated with 25HC3S or salt thereof in accordance with the present disclosure exhibit decreased liver cell apoptosis as compared to receiving no treatment or standard of care.

In some cases, the patient to be treated is identified by plasma or serum biomarkers, including inflammatory, cell death, and fibrosis markers, as measured by adiponectin, high sensitivity C-reactive protein (hsCRP), cytokines (such as interleukin (IL)-1β, IL-6, IL-12, IL-17, IL-18, and tumor necrosis factor alpha (TNFα)), cytokeratin-18 (both M30 and M65), N-terminal type III collagen propeptide (pro-C3), plasminogen activator inhibitor-1 (PAI1), serum bile acids, tissue inhibitor of matrix metalloproteinases-1 (TIMP1), and/or hyaluronic acid (HA).

In embodiments, the patient to be treated may be a human subject including newborns, infants, children and adults. In some embodiments, a patient to be treated is a human adult. In some embodiments, a patient to be treated is a child. In some embodiments, the patient is a human subject that is aged 17 years or younger, such as 15 years or younger, such as 10 years or younger, such as 9 years or younger, such as 8 years or younger, such as 7 years or younger, such as 6 years or younger, such as 5 years or younger, such as 4 years or younger, such as 3 years or younger, such as 2 years or younger, such as 1 year or younger, such as 6 months or younger, such as 1 month or younger and including a newborn human subject. In some embodiments, the patient is a human subject that is from age 18 to 44 years, such as from age 20 to 40 years, such as from age 25 to 35 years. In some embodiments, the patient is a human subject that is from age 45 to 65 years, such as from age 50 to 60 years. In certain embodiments, the patient is a human subject that is age 65 years or older, such as age 70 years or older, such as age 75 years or older, such as age 80 years or older, such as age 85 years or older, such as age 90 years or older, such as age 95 years or older and including a human subject that is age 100 years or older.

In some cases, the patient to be treated has a BMI >20 kg/m², such as >25 kg/m², >30 kg/m², or >35 kg/m². In some cases, the patient to be treated has a BMI ranging from about 20 kg/m² to about 60 kg/m², such as about 25 kg/m² to about 50 kg/m², or about 30 kg/m² to about 40 kg/m².

In some embodiments, a patient to be treated does not have alcoholic hepatitis (AH). In some embodiments, a patient to be treated does not have COVID-19.

For the avoidance of doubt, patients to be treated can have a plurality of two or more of the above features and the present disclosure expressly includes methods of treatment that are carried out on patients having any combination of these features. One strictly non-limiting combination of such diagnostic features is the combination is features defined as the inclusion criteria in the Example section of this specification (particularly inclusive criteria 4, but also other numbered criteria or any combination of these numbered criteria).

### Dosing Regimens

Implementation of the methods generally involves identifying patients suffering from NASH and administering 25HC3S or salt thereof in an acceptable form by an appropriate route.

In some embodiments, the total amount of 25HC3S or salt thereof administered ranges from 1 mg/day to 100 mg/day, such as about 10 mg/day to about 80 mg/day or about 30 mg/day to about 70 mg/day (e.g. about 40 to about 60 mg/day or about 50 mg/day).

In some cases, the total amount per kg of body weight of 25HC3S or salt thereof that is administered to the subject ranges from about 0.1 mg/kg/day to about 5 mg/kg/day, such as from about 0.2 mg/kg/day to about 4 mg/kg/day, about 0.3 mg/kg/day to about 3 mg/kg/day, or about 0.4 mg/kg/day to about 2 mg/kg/day.

The 25HC3S or salt thereof to be administered in the methods can be administered in one dose or in a plurality of separate doses over a period of time (also referred to herein as a “dosing period”). In some cases, the administering comprises administering a plurality of doses of the 25HC3S or salt thereof.

In some cases, the doses are administered at a frequency ranging from once weekly to three times a day. In some cases, the doses are administered once a day. In some cases, the doses are administered twice a day.

The administration of the compound of the present disclosure may be intermittent, or at a gradual or continuous, constant or controlled rate. Administration may be through any route, such as oral, transdermal, or parenteral, including injection intravenously, intramuscularly, and/or subcutaneously. Oral administration is typically preferred.

In the present disclosure the 25HC3S or salt thereof is administered in a specified amount defined in mg/day. However, as will be clear to those of ordinary skill in the art, the disclosure embraces dosage regimens in which the administering comprises administering a plurality of doses of the 25HC3S or salt thereof and in which the frequency of administration can be a plurality of times a day or less than once a day. For the avoidance of doubt, therefore, it will be appreciated that the specified amount in mg/day refers to the mean average total amount of 25HC3S or salt thereof that is administered per day in a dosing period (wherein the dosing period typically commences on the first day of administration of the recited daily dose in mg/day, and therefore optionally excluding any preliminary period of dose escalation). For instance, if doses are administered twice a day, then the specified amount defined in mg/day is equal to the total amount in mg of the two doses. As a further example, if the doses are administered once weekly, then the specified amount defined in mg/day is equal to one seventh of the dose that is administered once weekly. The dosing period typically terminates on the final day of administration of the recited daily dose in mg/day. In general, therefore, the recited daily dose in mg/day is the total amount of 25HC3S or salt thereof that is administered in the dosing period, divided by the number of days in the dosing period.

In embodiments of the disclosure, the orally administering comprises administering said amount for a dosing period of at least 7 days, such as at least 14 days, or at least 28 days, including at least 56 days, at least 3 months, at least 6 months, or at least 1 year. In embodiments, the dosing period continues until the treatment is determined to have resulted in an improvement in one or more parameters such as improved ALT enzyme levels, decreased inflammation, decreased steatosis, reduced severity of NASH symptoms, reduced levels of NASH biomarkers such as CK-18, or the slowing, stopping, or improving liver fibrosis, as further discussed herein.

In some cases, the subject is taking a lipid lowering drug, such as at least one of a statin, fenofibrate, omega-3 fatty acid, icosapent ethyl, and fish oil, or the method further comprises administering a lipid lowering drug, such as at least one of a statin, fenofibrate, omega-3 fatty acid, icosapent ethyl, and fish oil, to the subject. For instance, the method may further comprise administering at least one of atorvastatin, fluvastatin, lovastatin, pitavastatin, pravastatin, rosuvastatin, and simvastatin. The results of administering both 25HC3S or salt thereof and a statin are surprising with respect to the degree of reduced triglycerides and non-HDL cholesterol.

The results of administering 25HC3S or salt thereof in subjects receiving statin therapy are surprising, for instance with respect to the degree of reduced deleterious symptoms, e.g. reduced triglycerides and non-HDL cholesterol. Statin therapy comprises administration of at least one statin, such as but not limited to at least one of atorvastatin, fluvastatin, lovastatin, pitavastatin, pravastatin, rosuvastatin, and simvastatin. For avoidance of doubt, the term a “human subject [is] receiving statin therapy”, when defining a subject to be treated according to the disclosure, typically refers to a human subject who is receiving/has received statin therapy prior to commencing a method of the disclosure, e.g. prior to commencing administration of the 25HC3S or salt thereof. The human subject may or may not continue to receive statin therapy during the period of treatment with the 25HCS3 or salt thereof, and any continued statin therapy may be identical to or different from the statin therapy prior to commencing the period of treatment with the 25HCS3 or salt thereof. As would be well known to those skilled in the art, statin therapy is a widely prescribed medical therapy and hence identifies a readily recognisable and unambiguously defined grouping of human subjects (i.e., a specific patient group) on which the subject-matter of the present disclosure can beneficially be practised.

In some cases, the time to maximum drug concentrations (Tₘₐₓ) after administration of the 25HC3S or salt thereof ranges from 1 hour to 5 hours, such as 1.5 hours to 4 hours or 2 hours to 3.5 hours.

In some cases, the subject exhibits a Cmax of 25HC3S ranging from about 25 ng/mL to about 200 ng/mL, from about 50 ng/mL to about 150 ng/mL, or from about 75 ng/mL to about 125 ng/mL. The subject may exhibit a Cmax of 25HC3S ranging from about 100 ng/mL to about 300 ng/mL, from about 120 ng/mL to about 250 ng/mL, or from about 150 ng/mL to about 200 ng/mL, per 100 mg of orally administered 25HC3S or salt thereof.

In some cases, the subject exhibits an AUCinf of 25HC3S ranging from about 300 ng*h/mL to about 1000 ng*h/mL, about 400 ng*h/mL to about 900 ng*h/mL, or from about 500 ng*h/mL to about 800 ng*h/mL. The subject may exhibit an AUCinf of 25HC3S ranging from about 600 ng*h/mL to about 1000 ng*h/mL, about 700 ng*h/mL to about 900 ng*h/mL, or from about 800 ng*h/mL to about 900 ng*h/mL, per 100 mg of orally administered 25HC3S or salt thereof.

In some cases, the subject exhibits an apparent volume of distribution (Vz/F) of 25HC3S ranging from about 300 L to about 1000 L, about 400 L to about 900 L, or from about 500 L to about 800 L.

In some cases, the subject exhibits an apparent clearance (CL/F) of 25HC3S ranging from about 100 L to about 200 L/h, about 110 L/h to about 180 L/h, or from about 120 L/h to about 160 L/h.

In some embodiments, the treatment results in an improvement in one or more parameters such as improved ALT enzyme levels, decreased inflammation, decreased steatosis, reduced severity of NASH symptoms, reduced levels of NASH biomarkers such as CK-18, or the slowing, stopping, or improving liver fibrosis.

In some embodiments, treatment results in a reduction in hepatocyte ballooning in the subject. In some embodiments, treatment results in decreased inflammation and/or fibrosis in a NASH patient. In some embodiments, treatment results in a reduction in plasma CK-18 levels in the subject.

In some embodiments, treatment according to the methods described herein results in an improvement in one or more parameters such as, but not limited to, an improvement in NAS (ballooning and inflammation) and/or fibrosis; an improvement in SAF (steatosis, activity, and fibrosis) score; complete resolution of steatohepatitis; no worsening of fibrosis; an improvement in fibrosis without a worsening of steatohepatitis; or an increased time to disease progression as measured by histopathologic assessment of progression to cirrhosis, death, liver transplant, hepatocellular carcinoma, and decompensation events such as hepatic encephalopathy, variceal bleeding requiring hospitalization, ascites requiring intervention, and spontaneous bacteria peritonitis. In some embodiments, treatment according to the methods described herein results in an improvement (i.e., a reduction) in hepatocyte ballooning. In some embodiments, hepatocyte ballooning is visualized using hematoxylin and eosin straining.

In some embodiments, treatment according to the methods described herein results in an improvement in one or more biomarkers of NASH, such as but not limited to markers of apoptosis (e.g., CK-18 fragments), adipokines (e.g., adiponectin, leptin, resistin, or visfatin), inflammatory markers (e.g., TNF-a, IL-6, chemo-attractant protein-1, or high sensitivity C-reactive protein). See, e.g., Neuman et al., Can J Gastroenterol Hepatol, 28:607-618 (2014); Castera et al., Nat Rev Gastroenterol Hepatol., 10:666-675 (2013). In some embodiments, biomarker values are measured using a sample that comprises a fluid, e.g., blood, plasma, serum, urine, or cerebrospinal fluid. In some embodiments, biomarker values are measured using a sample that comprises cells and/or tissues, e.g., hepatocytes or liver tissue. In some embodiments, treatment results in an improvement in the biomarker CK-18. In some embodiments, treatment results in a reduction in plasma CK-18 levels in the subject.

In some embodiments, a patient is monitored during the course of 25HC3S or salt thereof therapy using a diagnostic test as described herein (e.g., using abdominal imaging tests). In some embodiments, the method further comprises continuing a course of therapy (e.g., a dosage of 25HC3S or salt thereof as described herein). In some embodiments, the method further comprises tapering, reducing, or stopping the administered amount of 25HC3S or salt thereof if the diagnosis warrants, e.g., when a cure is effected, a lower dose appears to be safer or equally efficacious as a higher dose, or no continuing therapeutic effect is expected. In some embodiments, the methods can comprise increasing the administered amount of 25HC3S or salt thereof if it is determined not to be efficacious, as well as stopping therapy if it is determined dose escalation or continued dosing at any dose is unlikely to be efficacious.

In some embodiments where the patient is undergoing treatment in accordance with the present disclosure, indications of NASH by abdominal imaging, ultrasound examination, magnetic resonance imaging, CT scan, and/or biopsy may be less than those measured in the patient prior to treatment, which is indicative that the patient is responding positively to the therapy. In cases where the patient is responding positively to a therapy of the present disclosure, the therapy is continued until the presence of the condition is reduced to a level comparable to a normal control level. Optionally, the therapy is continued to maintain alleviation of NASH symptoms. Alternatively, the therapy is continued until a desired level of steatosis is achieved in the patient (including the absence of steatosis). Treatment may be continued for so long as it is determined to be efficacious using assessment by abdominal imaging, ultrasound examination, magnetic resonance imaging, CT scan, and/or biopsy. The treatment may be determined to be efficacious through measured improvement in one or more of steatosis, ballooning, and necroinflammation. In one embodiment, the treatment is determined to be efficacious through measured improvement indicated by induced reduction in ballooning. In one embodiment, the treatment is determined to be efficacious through measured improvement indicated by a reduction in inflammation. In one embodiment, the treatment is determined to be efficacious through measured improvement indicated by at least one of reduced serum ALT levels, improved insulin sensitivity (e.g., reduced insulin resistance), reduced steatosis, reduced inflammation, and reduced fibrosis. In one embodiment, the treatment is determined to be efficacious through measured improvement indicated by induced regression or reversal of fibrosis and/or cirrhosis.

In some embodiments, treatment results in an improvement in one or more parameters (e.g., a reduction in NAS or SAF score, a reduction in hepatocyte ballooning, a reduction in fibrosis, or a reduction in CK-18 levels) of at least 10%, at least 20%, at least 30%, at least 40%, at least 50%, at least 60%, at least 70%, at least 80%, or at least 90% as compared to a control value. In some embodiments, treatment results in an improvement in one or more parameters of at least 2-fold, at least 3-fold, at least 4-fold, at least 5-fold, at least 6-fold, at least 7-fold, at least 8-fold, at least 9-fold, or at least 10-fold as compared to a control value. In some embodiments, the control value is a baseline value for the subject that is determined prior to the onset of treatment. In some cases, treatment showed ≥10% liver fat reduction from baseline as measured by MRI-PDFF.

In some embodiments, the present disclosure provides methods of determining efficacy of a NASH treatment in a subject in need thereof by (a) measuring the level and severity of NASH via abdominal imaging, ultrasound examination, magnetic resonance imaging, CT scan, and/or liver biopsy in a subject in need thereof, where the level and severity of NASH is measured after treatment has started, (b) comparing the level and severity of NASH as measured in step (a) to a baseline level and severity of NASH, where the baseline level and severity is measured in the same subject before treatment is begun, and (c) determining the efficacy of the NASH treatment based on the comparison step.

Furthermore, in some embodiments the present disclosure provides methods of determining efficacy of a NASH treatment in a subject in need thereof by (a) measuring the level and severity of NASH in a subject in need thereof after treatment has begun, (b) comparing the level and severity of the NASH to a reference value, where the reference value represents an average value determined from a population of patients suffering from NASH, and (c) determining the efficacy of the NASH treatment based on the comparison step. In some embodiments, efficacy of therapy is determined by liver biopsy and analysis to evaluate NAFLD Activity Score (NAS) and fibrosis; the transjugular liver biopsy method can be employed for this purpose. Suitable patients include patients with biopsy proven NASH, patients at high risk for NASH, patients with a NAS greater than or equal to 4, NASH patients with liver fibrosis, and NASH patients with liver fibrosis of stage 2 or greater.

In some embodiments, patients responding to therapy in accordance with the invention are expected to show at least a slowing of any increase in CK-18 levels as therapy continues. In some embodiments, those patients responding most favorably to therapy will have CK-18 levels that stabilize and decline over time as full therapeutic benefit is realized. Thus, in some embodiments, the present disclosure provides methods of determining efficacy of a NASH treatment in a subject in need thereof by (a) measuring the level and severity of NASH via measuring the level of the biomarker CK-18 in a sample from the sample from the subject (e.g., a blood, plasma, or tissue sample), wherein the level and severity of NASH is measured after treatment has started, (b) comparing the level and severity of NASH measured in (a) to a baseline level and severity of NASH in the subject that is measured in the same subject before treatment is begun, and (c) determining the efficacy of the NASH treatment based on the comparison step; wherein a plateau or decrease in CK-18 levels is indicative of efficacy of the NASH treatment.

In some embodiments, patients treated in accordance with the invention exhibit levels of one or more biomarkers that decline over time as full therapeutic benefit is realized. Thus, in some embodiments, the present disclosure provides methods of determining efficacy of a NASH treatment in a subject in need thereof by (a) measuring the level and severity of NASH via measuring the level of one or more biomarkers selected from the group consisting of C-reactive protein, plasminogen activator inhibitor-1, interleukin-1 beta, interleukin-6, interleukin-12, interleukin-17, interleukin-18, tumor necrosis factor, bile acid, adiponectin and adiponectin, HMW; (b) comparing the level and severity of NASH measured in (a) to a baseline level and severity of NASH in the subject that is measured in the same subject before treatment is begun, and (c) determining the efficacy of the NASH treatment based on the comparison step; wherein a plateau or decrease in the biomarker level(s) is indicative of efficacy of the NASH treatment.

In some instances, methods include treating a subject in a manner sufficient to reduce the level of a CK-18, M30 biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of a CK-18, M65 biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of a C-reactive protein biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of a plasminogen activator inhibitor-1 biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of an interleukin-1 beta biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of an interleukin-6 biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of an interleukin-12 biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of an interleukin-17 biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of an interleukin-18 biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of a tumor necrosis factor biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of a bile acid biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of an adiponectin biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In some instances, methods include treating a subject in a manner sufficient to reduce the level of an adiponectin, HMW biomarker, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including by 40% or more.

In certain embodiments, methods and compositions of the present disclosure are sufficient to reduce the amount of one or more elevated serum liver enzymes. In some instances, the subject methods and compositions are sufficient to reduce serum alanine aminotransferase (ALT), such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum ALT by 40% or more. In certain instances, administering 25HC3S is sufficient to reduce the amount of serum ALT to an amount that is below the upper limit of normal levels of ALT.

In certain embodiments, methods and compositions of the present disclosure are sufficient to reduce the amount of one or more elevated serum liver enzymes. In some instances, the subject methods and compositions are sufficient to reduce serum aspartate aminotransferase (AST), such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum AST by 40% or more. In certain instances, administering 25HC3S is sufficient to reduce the amount of serum AST to an amount that is below the upper limit of normal levels of AST.

In some instances, the subject methods and compositions are sufficient to reduce serum gamma glutamyl transpeptidase (GGT), such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more, such as by 16% or more, such as by 17% or more, such as by 18% or more, such as by 19% or more, such as by 20% or more, such as by 25% or more, such as by 30% or more, such as by 35% or more and including reducing the presence of serum GGT by 40% or more. In certain instances, administering 25HC3S is sufficient to reduce the amount of serum GGT to an amount that is below the upper limit of normal levels of GGT.

In some instances, the subject methods and compositions are sufficient to reduce liver stiffness, as measured by FibroScan, an ultrasound machine that measures the stiffness of liver tissue, such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more. In certain instances, administering 25HC3S is sufficient to reduce the amount of liver stiffness to an amount that is below the upper limit of normal levels of liver stiffness.

In some instances, the subject methods and compositions are sufficient to reduce serum triglyceride (TG), such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more. In certain instances, administering 25HC3S is sufficient to reduce the amount of serum TG to an amount that is below the upper limit of normal levels of TG.

In some instances, the subject methods and compositions are sufficient to reduce serum low-density lipoprotein—cholesterol (LDL-C), such as by 1% or more, such as by 2% or more, such as by 3% or more, such as by 4% or more, such as by 5% or more, such as by 6% or more, such as by 7% or more, such as by 8% or more, such as by 9% or more, such as by 10% or more, such as by 11% or more, such as by 12% or more, such as by 13% or more, such as by 14% or more, such as by 15% or more. In certain instances, administering 25HC3S is sufficient to reduce the amount of serum LDL-C to an amount that is below the upper limit of normal levels of LDL-C.

### Compositions, Unit Dosage Forms, and Kits

The 25HC3S may be administered in the pure form or in a pharmaceutically acceptable formulation including suitable elixirs and the like (generally referred to a “carriers”) or as pharmaceutically acceptable salts (e.g., alkali metal salts such as sodium, potassium, calcium or lithium salts, ammonium, etc.) or other complexes. In some instances, the 25HC3S is administered as a salt of 25HC3S, such as the sodium salt of 25HC3S. The 25HC3S or salt thereof is typically administered as compositions that are suitable for oral, injection and/or intravenous administration.

The active ingredients may be mixed with excipients which are pharmaceutically acceptable and compatible with the active ingredients, e.g., pharmaceutically and physiologically acceptable carriers. Suitable excipients include, for example, water, saline (sodium chloride), a cyclic oligosaccharide (such as cyclodextrin, for example those described in U.S. Patent Publication No. 2019/0269695, the disclosure of which is herein incorporated by reference e.g., hydroxypropyl-beta-cyclodextrin), dextrose, glycerol, ethanol and the like, or combinations thereof. In addition, the composition may contain minor amounts of auxiliary substances such as wetting or emulsifying agents, pH buffering agents (e.g., phosphate buffer), and the like. Water may be used as the carrier for the preparation of compositions (e.g., injectable compositions), which may also include conventional buffers and agents to render the composition isotonic. Other potential additives and other materials (preferably those which are generally regarded as safe [GRAS]) include: surfactants (TWEEN®, oleic acid, etc.); solvents, stabilizers, elixirs, and encapsulants (lactose, liposomes, etc). Preservatives such as methyl paraben or benzalkium chloride may also be used. The composition of the present disclosure may contain any such additional ingredients so as to provide the composition in a form suitable for the intended route of administration. In addition, the compounds may be formulated with aqueous or oil based vehicles.

Depending on the formulation, it is expected that the 25HC3S or salt thereof will be present at about 1 wt % to about 99 wt % of the composition and the vehicular “carrier” will constitute about 1 wt % to about 99 wt % of the composition. The pharmaceutical compositions of the present disclosure may include any suitable pharmaceutically acceptable additives or adjuncts to the extent that they do not hinder or interfere with the therapeutic effect of the 25HC3S or salt thereof.

In one aspect, compositions, unit dosage forms, pharmaceutical packages, and kits comprising 25HC3S or salt thereof for use in the methods described herein are provided. In some embodiments, the formulations, unit dosage forms, pharmaceutical packages, and/or kits are for use in treating NASH.

In some embodiments, oral 25HC3S or salt thereof formulations are formulated as immediate release preparations, and are conveniently packaged, for example, in unit dosage forms in the form of a pill, capsule, or tablet, which in turn may be in a pill bottle or blister packaging. Dosages and desired drug concentration of pharmaceutical compositions of the disclosure may vary depending on the particular use envisioned. The determination of the appropriate dosage or route of administration is well within the skill of one in the art.

In some embodiments, formulations are formulated as sustained release preparations, and are conveniently packaged, for example, in unit dosage forms in form of a vial, ampoule, syringe, bottle or other liquid compatible containers.

In some embodiments, the pharmaceutical package or kit is for use in treating NASH. In some embodiments, the pharmaceutical package or kit further comprises instructional materials for use according to a method disclosed herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD-ROM), and the like. Such media may include addresses to internet sites that provide such instructional materials.

The present invention will be further illustrated by way of the following Examples. These Examples are non-limiting and do not restrict the scope of the invention. Unless stated otherwise, all percentages, parts, etc. presented in the Examples are by weight.

### EXAMPLE 1

### Overview

The present Example was a randomized, open label, multi center US study to evaluate safety, pharmacokinetics, and signals of biological activity of 4-week administration of 25HC3S in NASH patients with stage 1-3 fibrosis. A total of 63 patients completed the study with 21 patients per dose group (completion of MRI-PDFF measurement). 25HC3S sodium was orally administered daily at 50 mg, 150 mg, or 600 mg (300 mg BID). The patients in this trial were monitored for 2 weeks (14 days), dosed for 4 weeks (28 days), and followed up for an additional 4 weeks (28 days).
Title of Trial:
   A Randomized, Open-label, Phase lb Study to Evaluate Safety, Pharmacokinetic and Pharmacodynamic Signals of 25HC3S in Patients with Non-Alcoholic Steatohepatitis (NASH)
Phase of Development:
   Phase 1b
Endpoints:
   To determine the safety and pharmacokinetics (PK) of 4 week daily oral dosing of 25HC3S in subjects with NASH
   To determine the effect of 25HC3S on pharmacodynamic (PD) signals in subjects with NASH
      Change of hepatic fat content from baseline* to end of dosing (end of Week 6) as measured by magnetic resonance imaging-proton density fat fraction (MRI-PDFF)
      Change of hepatic stiffness from baseline to end of dosing (end of Week 6) as measured by transient elastography (TE)
      Liver function parameters as measured by plasma alanine aminotransferase (ALT), aspartate aminotransferase (AST), and gamma-glutamyl transpeptidase (GGT) from baseline to end of dosing (end of Week 6), weekly during the 4 weeks of dosing, and end of the study (end of Week 10); the panel will be part of laboratory safety tests
      Metabolic panels as measured by serum cholesterol, low density lipoprotein cholesterol (LDL-C), high density lipoprotein cholesterol (HDL) and triglycerides (TG) from baseline to end of dosing (end of Week 6), weekly during the 4 weeks of dosing, and end of the study (end of Week 10)
   *: Baseline is defined as the last non-missing value before the first dose of study drug
Safety Assessments:
   Adverse Events (AEs) were recorded from the time of signing the informed consent form through the end of study or early termination visit respectively:
   Vital Signs, Physical Examination and 12-lead ECG findings
   Safety Laboratory Tests (chemistry, hematology, coagulation and urinalysis)
Trial Population:
   A total of 65 subjects (including both male and female), diagnosed with NASH or suspected NASH, were enrolled in the study. Each of the below dose groups included at least 20 patients.
      Group 1: 50 mg 25HC3S sodium, oral QD
      Group 2: 150 mg 25HC3S sodium, oral QD
      Group 3 : 300 mg 25HC3S sodium, oral BID
Inclusion Criteria:
   1. Subjects provided written informed consent to participate in the study
   2. Males or females subjects 18 years or older, at the time of signing informed consent
   3. BMI 20-45 kg/m²
   4. Subjects had an historic histologic diagnosis of NASH, confirmed during the 12 months prior to the screening visit, demonstrating the presence of both Stage 1-3 fibrosis and a NAS≥4, with at least 1 point for each of the three components (steatosis, hepatocellular ballooning, and lobular inflammation), OR possessed a diagnosis of ‘suspected NASH’, using a combination of a clinical diagnosis*, laboratory results, and imaging assessments of steatosis (including MRI-PDFF>10% and CAP score>238 dB/m on Fibroscan® for heterogeneous livers) and fibrosis, the latter being defined in this clinical trial as:
      a. Value of Fibroscan® ≥7 kPa
      OR
      b. MRE≥2.75 kPa
   *Clinical diagnosis of NASH was defined in this clinical trial as the existence of one or more of the following risk factors for NASH, namely:
      a. Type 2 diabetes or elevated fasting blood sugar
      b. Abdominal obesity
      c. Elevated lipid levels, especially elevated levels of serum triglycerides
      d. Hypertension, or
      e. Low levels of HDL cholesterol
   5. For serum transaminases, ALT concentration for all patients at the time of screening was >1 and <5 times upper limit of normal (ULN) of the central lab in the absence of another cause of liver disease. If a patient had lab records in the past 6 months, ALT concentrations were <20 U/L female and <30 U/L male. AST concentration for all patients at the time of screening was <5x ULN
   6. Serum ALT, AST, ALP, and TBL concentrations did not fluctuate >30% during the screening period
   7. Platelet counts ≥120,000/mm³
   8. Female subjects were eligible for the study if they met the following criteria:
      Are not pregnant or nursing
      Women of child-bearing potential (defined as females who are not surgically sterile or who are not over the age of 52 and amenorrheic for at least 12 months) must utilize appropriate birth control throughout the study duration. Acceptable methods that may be used are abstinence, birth control pills (“The Pill”) or patch, diaphragm, IUD (coil), vaginal ring, condom, surgical sterilization or progestin implant or injection, or sexual activity limited to a sterile (e.g., vasectomized) male partner
   9. Male participants agreed to consistently and correctly use a condom in combination with one of the above methods of birth control from enrolment to 30 days after the last dose of study medication
   10. Participants were able to comply with dosing and able to complete the study schedule of assessments

### Results

25HC3S was well tolerated at all three doses with no drug related serious adverse events observed. PK parameters after repeat dosing were comparable to those after a single dose and were dose dependent.

Low and high dose groups showed statistically significant median reductions from baseline of serum ALT levels at −16% and −17%, respectively. The high dose group also showed statistically significant median reductions from baseline of serum AST (−18%) and GGT (−8%) levels, as well as FIB-4 (−15%) and APRI (−26%) scores. The low dose group had a statistically significant reduction at day 28 from baseline in liver stiffness as measured by Fibroscan (−10%).

Patients in the low and medium dose groups also had statistically significant median reduction at day 28 from baseline of serum triglycerides (−13% in the 50 mg group) or LDL-C (−11% in the 150 mg group). Patients with elevated baseline triglycerides (≥200 mg/dL; n=16) across all dose groups had a median reduction at day 28 from baseline of −24% (p<0.01).

In each dose group, 43% of patients who underwent MRI-PDFF, after 4-week dosing, showed ≥10% liver fat reduction from baseline as measured by MRI-PDFF. The median reduction from baseline of liver fat in these patients in each sub-group, −18%, −19%, and −23%, respectively, were statistically significant. The reduction of liver fat content of each dose group was also accompanied by significant reduction of serum ALT levels. Each sub-group showed statistically significant median reduction from baseline of serum ALT levels at −21%, −19%, and −32%, respectively.

There was a 24% reduction in serum triglycerides in patients with elevated baseline triglycerides (≥200 mg/dL; n=16) across all dose groups at day 28 from baseline (p<0.01).

In the 43% of patients with ≥10% liver fat reduction by PDFF, both low and high dose 4-week 25HC3S treated patients also had statistically significant median reductions of AST (−24% and −39%), FIB-4 scores (−19% and −21%) and APRI scores (−27% and −36%), while the low dose treated patients also had a statistically significant median reduction of GGT (−13%) levels.

In addition, there were trended or statistically significant reductions of liver stiffness as measured by Fibroscan® in the 43% patients with ≥10% liver fat reduction by PDFF in all 3 dose groups (−7%, −9%, and −9%, respectively).

The results are summarized further in the following Tables:

### Top Line Data Summary (Day 28 vs Baseline)

For all tables below, *indicates p-value <0.05; **indicates p<0.01; ***indicates p<0.001

| Median | $\frac{50 mg QD}{( n = 21 ‐ 23 )}$ | $\frac{150 mg QD}{( n = 20 ‐ 21 )}$ | $\frac{300 mg BID}{( n = 20 ‐ 21 )}$ |
|---|---|---|---|
| ALT | −16%* | −10% | −17%*** |
| | (n = 22) | (n = 20) | (n = 20) |
| AST | −14% | −9% | −18%** |
| | (n = 22) | (n = 20) | (n = 20) |
| GGT | −6% | −1% | −8%* |
| | (n = 23) | (n = 20) | (n = 21) |
| LDL-C | −6% | −11%* | −7% |
| | (n = 22) | (n = 20) | (n = 21) |
| Non-HDL-C | −8% | −5% | −1% |
| | (n = 23) | (n = 20) | (n = 21) |
| Triglycerides | −13%* | −3% | −2% |
| | (n = 23) | (n = 20) | (n = 21) |
| Platelet | +2% | +4% | +7% |
| | (n = 22) | (n = 20) | (n = 19) |
| CK18, M30 | −14.6% | −8.6% | −16.1% |
| CK18, M65 | −18.1% | −9.9% | −35.0% |

ALT = alanine aminotransferase;
AST = aspartate aminotransferase;
GGT = Gamma-glutamyl transferase;
LDL-C (Low-Density Lipoprotein-Cholesterol);
Non-HDL-C (Total cholesterol excluding High-Density Lipoprotein-Cholesterol);
QD (once a day);
BID (twice a day)

Non-Invasive Fibrosis Scores

| Median | 50 mg QD | 150 mg QD | 300 mg BID |
|---|---|---|---|
| FIB-4 | −6% | −4% | −15%** |
| APRI | −14% | −7% | −26%*** |

FIB 4 score is a non-invasive liver fibrosis assessment based on patient age, platelet count, AST and ALT values.
APRI (aspartate aminotransferase to platelet ratio index) is one of many different kinds of tests that are used to measure the levels of fibrosis and, in turn, cirrhosis of the liver.

Non-invasive Imaging

| Median | 50 mg QD | 150 mg QD | 300 mg BID |
|---|---|---|---|
| MRI-PDFF | −7% | −7% | −4% |
| | (n = 21) | (n = 21) | (n = 21) |
| Fibroscan | −10%** | −9% | −1% |
| | (n = 22) | (n = 20) | (n = 21) |

MRI-PDFF is Magnetic Resonance Imaging - Proton Density Fat Fraction is a non-invasive measure of the proportion of liver tissue which is composed of fat.
FibroScan is a specialized ultrasound machine that measures the stiffness of liver tissue.

The following tables show Day 28 vs Baseline data in patients who had ≥10% reduction in MRI-PDFF

Clinical Chemistry
Patients with ≥10% Reduction in MRI-PDFF

| Median | $\frac{50 mg QD}{( n = 9 )}$ | $\frac{150 mg QD}{( n = 8 )}$ | $\frac{300 mg BID}{( n = 9 )}$ |
|---|---|---|---|
| ALT | −21%** | −19%* | −32%*** |
| AST | −24%** | −21% | −39%*** |
| GGT | −13%*** | −16%* | −14% |
| LDL-C | −7% | −11% | −8%* |
| Non-HDL-C | −10% | −8%* | −12%* |
| Triglycerides | −9% | 0% | −8% |
| Platelet | +6%* | −2% | +2% |
| CK18, M30 | −22.8%*** | −3.8% | −42.1%* |
| CK18, M65 | −28.1%*** | −8.7% | −55.8%* |

Non-Invasive Fibrosis Scores
Patients with ≥10% Reduction in MRI-PDFF

| Median | 50 mg QD | 150 mg QD | 300 mg BID |
|---|---|---|---|
| FIB-4 | −19%** | −6% | −21%*** |
| APRI | −27%*** | −16% | −36%*** |

Non-Invasive Imaging
Patients with ≥10% Reduction in MRI-PDFF

| Median | $\frac{50 mg QD}{( n = 9 )}$ | $\frac{150 mg QD}{( n = 9 )}$ | $\frac{300 mg BID}{( n = 9 )}$ |
|---|---|---|---|
| MRI-PDFF | −18%*** | −19%*** | −23%*** |
| Fibroscan | −7% | −9%** | −9% |

Biomarkers
% Change from baseline at the end of dosing (median at Day 28)

| Biomarker | 50 mg QD | 150 mg QD | 300 mg BID |
|---|---|---|---|
| Cytokeratin 18, M30 | −14.6 | −8.6 | −16.1 |
| Cytokeratin 18, M65 | −18.1 | −9.9 | −35.0 |
| C Reactive Protein | −13.9 | −11.8 | 1.7 |
| Plasminogen Activator | −13.5 | −13.7 | −8.2 |
| Inhibitor-1 | | | |
| Interleukin-1 Beta | −0.1 | −0.6 | −0.2 |
| Interleukin-6 | −6.0 | 1.7 | 5.4 |
| Interleukin-12 | 0.0 | 0.0 | 0.0 |
| Interleukin-17 | −1.3 | −16.4 | −0.8 |
| Interleukin-18 | −8.9 | −5.0 | −2.1 |
| Tumor Necrosis Factor | −3.2 | −2.9 | −7.9 |
| Bile Acid | 0.0 | 0.0 | 1.6 |
| Adiponectin | −1.6 | −3.8 | 3.9 |
| Adiponectin, HMW | 0.0 | 1.0 | 1.0 |

### Pharmacokinetics

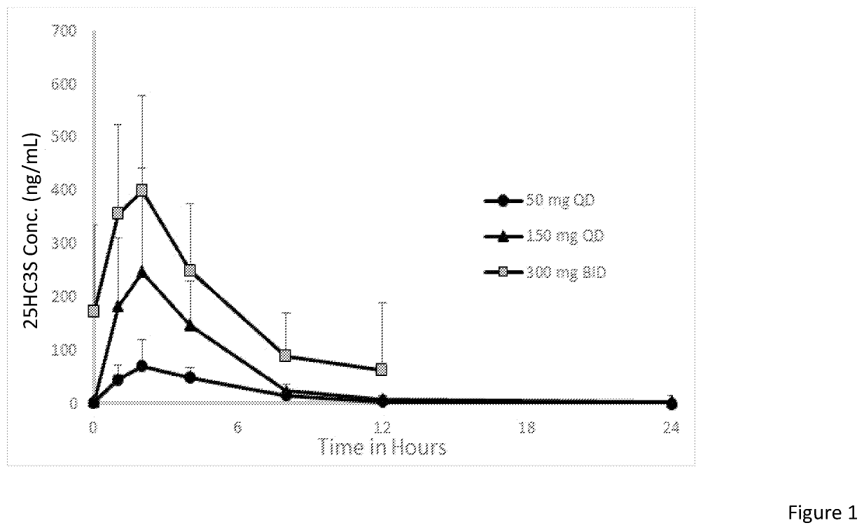

The pharmacokinetics of administered 25HC3S was determined. The mean (standard deviation) pharmacokinetic parameters are summarized in FIG. 1 and the table below.

### Reference of Pharmacokinetic Parameters

The following PK parameters were estimated for 25HC3S from the plasma concentration data.
Cₘₐₓ Maximum observed plasma concentration of 25HC3S
Tₘₐₓ The time (observed time point) of Cmax
Cₗₐₛₜ The last observed quantifiable concentration of 25HC3S in plasma
Tₗₐₛₜ The last observed time point of Ciast
AUC₀₋₁₂ Area under the plasma concentration versus time curve from time zero to 12 h post-dose. Calculated by the linear/log trapezoidal rule.
AUC₀₋ₗₐₛₜ Area under the plasma concentration versus time curve (linear/log trapezoidal rule) from time zero to the last measured concentration above the limit of quantitation.
Cₘᵢₙ Minimum observed concentration of 25HC3S.
Tₗₐₛₜ The last (observed time point) of Cₗₐₛₜ
AUC_{inf} The area under the concentration versus time curve (linear/log trapezoidal rule) extrapolated to infinite time, calculated as AUC₀₋ₗₐₛₜ+(Cₗₐₛₜ/λ)
% AUCₑₓₚ Percentage of AUC extrapolated between AUC₀₋ₗₐₛₜ and AUC_{inf}
T_{1/2} An estimate of the terminal elimination half-life of the drug in plasma, calculated by dividing the natural log of 2 by the terminal elimination rate constant (λ)
λ First order rate constant associated with the terminal log-linear portion of the plasma concentration versus time curve.
CL/F The apparent clearance after administration of the drug: CL=Dose/AUC_{inf}, where ‘Dose’ is the dose of the drug.
V_{z}/F The apparent volume of distribution of 25HC3S.

Pharmacokinetics

| Parameter | $\frac{50 mg QD}{( n = 22 )}$ | $\frac{150 mg QD}{( n = 21 )}$ | $\frac{300 mg BID}{( n = 21 )}$ |
|---|---|---|---|
| Cₘₐₓ (ng/mL) | 79.1 (45.1) | 273.5 (187.7) | 429.7 (167.7) |
| Tₘₐₓ (h) | 2.4 (1.0) | 2.0 (0.9) | 2.3 (2.4) |
| T_{1/2} (h) | 2.7 (1.4) | 2.7 (1.4) | 2.4 (1.0) |
| AUC_{(0-T)} (ng*h/mL) | 339.9 (113.9) | 1038.7 (542.5) | 2138.1 (1014.9) |
| CL/F (L/h) | 150.6 (51.5) | 176.6 (80.5) | 166.2 (60.4) |
| V_{z}/F (L) | 582.8 (338.2) | 669.1 (410.6) | 567.7 (297.1) |
| Metabolite/Drug Ratio | 0.04 (0.04) | 0.11 (0.03) | 0.12 (0.04) |

Prior to and during the study some of the subjects received a statin (atorvastatin, pravastatin, rosuvastatin, or simvastatin). The subjects receiving both 25HC3 and a statin had reduced triglycerides and non-HDL at day 28 after dosing as shown in the following Table:

| | | Mean | | Median |
|---|---|---|---|---|
| | Mean TG | Non-HDL | Median TG | Non-HDL |
| | to Baseline | to Baseline | to Baseline | to Baseline |
| Patients | Mean | Mean | Mean | Mean |
| All on a statin | −2% | −9% | −6% | −9% |
| (n = 20) | | | | |
| All on a statin | −10% | −11% | −9% | −10% |
| except one | | | | |
| outlier (n = 19) | | | | |

### Conclusion

The present Example showed that the low dose resulted in reduced liver fat (e.g., as measured by MRI-PDFF) compared with higher doses. The present Example showed that the medium dose resulted in improved low density lipoprotein cholesterol (LDL-C) levels. The present Example showed that the high dose resulted in improved enzyme levels (e.g., ALT, AST, GGT) suggesting improved liver function.

### EXAMPLE 2

### Overview

The present Example was a randomized, dose ranging, single dose safety and pharmacokinetic study of 25HC3S administered to subjects with NASH and control healthy subjects. This study was conducted in 2 successive cohorts evaluating 2 single-dose levels of oral 25HC3S. For each cohort, 10 subjects with NASH were enrolled which were further classified into cirrhotic and non-cirrhotic. Each subject received only one dose of study treatment. The second cohort was dosed after a review of safety and tolerability data from Cohort 1. Cohort 1 received 50 mg of 25HC3S sodium and Cohort 2 received 200 mg of 25HC3S sodium.

### Results

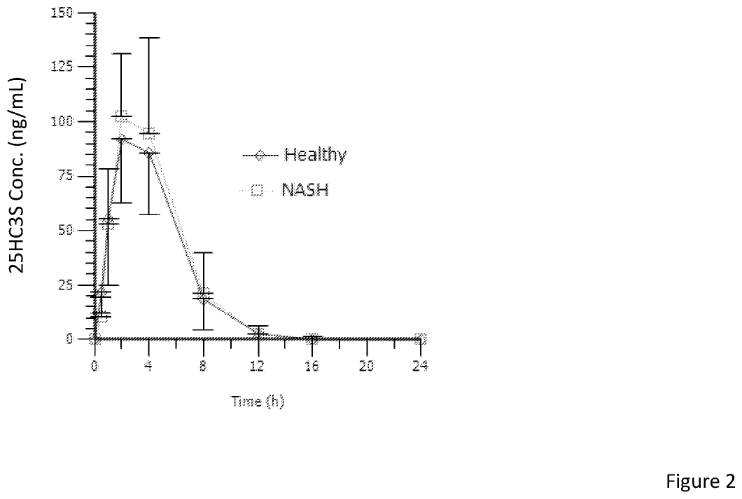

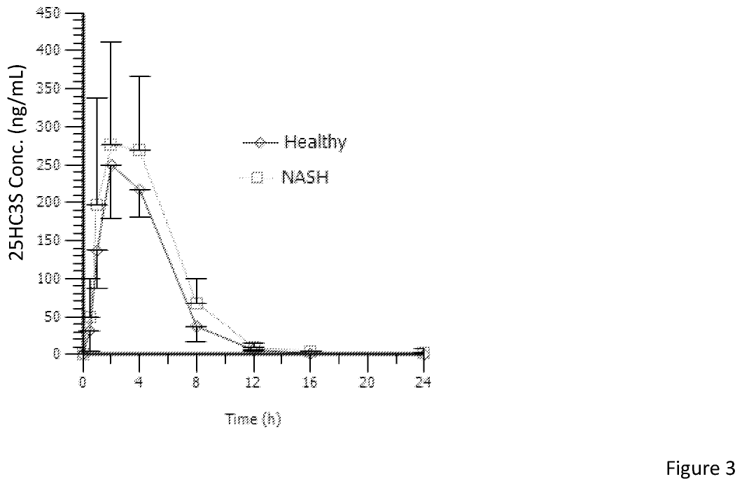

Pharmacokinetic (PK) plasma concentrations of 25HC3S are summarized in Table 2.1 (50 mg dose) and Table 2.2 (200 mg dose) below. Plasma 25HC3S levels were detectable up to 12 hours post-dose for Cohort 1 healthy subjects and up to 16 hours post-dose in Cohort 2 healthy subjects (Table 2.1). The plasma profiles were similar for both healthy and NASH subjects following administration of 50 mg 25HC3S (FIGS. 2) and 200 mg 25HC3S (FIG. 3). For healthy subjects, a four-fold increase in dose resulted in an approximate three-fold increase in mean Cₘₐₓ (50 mg dose: 93.967±27.343 ng/mL and 200 mg dose: 260.500±54.779 ng/mL). This was also observed for AUC parameters (Tables 2.1 and 2.2). Similarly, NASH subjects also displayed an approximate three-fold increase in both Cₘₐₓ and AUC parameters for a four-fold increase in dose (Tables 2.1 and 2.2). Mean % AUCₑₓₚ was low, suggesting that the blood sampling schedule was adequate to capture the majority of the AUC where it was possible to compute.

Individual plasma overlay plots indicated that despite differing subject numbers for healthy (n=6) and NASH (n=10) subjects, the NASH subjects tended to display a greater variability for Cₘₐₓ and AUC parameters. In NASH subjects, geometric mean Cmax increased by 18-24% over healthy subjects for Cohorts 1 and 2 which was accompanied by a 25-50% higher CV % geometric mean in NASH subjects (Table 2.1). Geometric mean AUC₀₋₁₂ and AUC₀₋ₗₐₛₜ were similar between NASH and healthy subjects in Cohort 1 but tended to be approximately 30% higher in Cohort 2, where the % CV was 50% higher in NASH subjects. AUC_{0-inf} (Cohort 2) was approximately 20% higher in NASH subjects. Hence, when the higher % CV was taken into account, no clear difference between healthy and NASH subjects in terms of Cₘₐₓ and AUC was concluded (Table 2.1).

TABLE 2.1
Cohort 1 (50 mg 25HC3S sodium): Pharmacokinetic Parameters

| Health | Subject | T_{1/2} | Tₘₐₓ | Cₘₐₓ | AUC₀₋₁₂ | AUC₀₋ₗₐₛₜ | AUC_{inf} | V_{z}/F | CL/F |
|---|---|---|---|---|---|---|---|---|---|
| Status | No. | (h) | (h) | (ng/mL) | (h*ng/mL) | (h*ng/mL) | (h*ng/mL) | (L) | (L/h) |
| Healthy | N | 2 | 6 | 6 | 4 | 6 | 2 | 2 | 2 |
| | Mean | 1.906 | 3.008 | 93.967 | 528.0 | 477.1 | 438.6 | 324.96 | 122.35 |
| | SD | 0.350 | 1.105 | 27.347 | 217.6 | 190.7 | 161.9 | 62.41 | 45.16 |
| | Geometric | 1.889 | 2.834 | 90.946 | 496.7 | 450.0 | 423.3 | 321.95 | 118.11 |
| | Mean | | | | | | | | |
| | CV % | 18.62 | 39.63 | 28.03 | 41.67 | 37.58 | 39.18 | 19.51 | 39.18 |
| | Geometric | | | | | | | | |
| | Mean | | | | | | | | |
| NASH | N | 5 | 10 | 10 | 5 | 10 | 5 | 5 | 5 |
| | Mean | 1.674 | 2.408 | 113.170 | 623.1 | 513.3 | 636.1 | 203.28 | 85.29 |
| | SD | 0.136 | 0.844 | 36.261 | 217.5 | 219.3 | 231.5 | 48.28 | 23.57 |
| | Geometric | 1.670 | 2.305 | 107.627 | 597.8 | 476.1 | 608.3 | 198.01 | 82.19 |
| | Mean | | | | | | | | |
| | CV % | 8.03 | 29.81 | 35.26 | 31.66 | 42.20 | 32.82 | 27.09 | 32.82 |
| | Geometric | | | | | | | | |
| | Mean | | | | | | | | |

TABLE 2.2
Cohort 1 (200 mg 25HC3S sodium): Pharmacokinetic Parameters

| Health | Subject | T_{1/2} | Tₘₐₓ | Cₘₐₓ | AUC₀₋₁₂ | AUC₀₋ₗₐₛₜ | AUC_{inf} | V_{z}/F | CL/F |
|---|---|---|---|---|---|---|---|---|---|
| Status | No. | (h) | (h) | (ng/mL) | (h*ng/mL) | (h*ng/mL) | (h*ng/mL) | (L) | (L/h) |
| Healthy | N | 6 | 6 | 6 | 6 | 6 | 6 | 6 | 6 |
| | Mean | 1.753 | 2.673 | 260.500 | 1175.9 | 1185.7 | 1194.3 | 434.05 | 171.25 |
| | SD | 0.388 | 1.028 | 54.779 | 189.4 | 192.4 | 192.1 | 128.07 | 28.60 |
| | Geometric | 1.719 | 2.528 | 255.698 | 1162.8 | 1172.5 | 1181.2 | 419.93 | 169.32 |
| | Mean | | | | | | | | |
| | CV % | 21.50 | 36.69 | 21.46 | 16.66 | 16.61 | 16.50 | 28.05 | 16.50 |
| | Geometric | | | | | | | | |
| | Mean | | | | | | | | |
| NASH | N | 7 | 10 | 10 | 10 | 10 | 7 | 7 | 7 |
| | Mean | 2.511 | 2.906 | 332.700 | 1541.0 | 1581.6 | 1428.5 | 540.23 | 143.19 |
| | SD | 1.751 | 1.198 | 99.454 | 417.9 | 413.9 | 247.0 | 457.19 | 21.67 |
| | Geometric | 2.196 | 2.647 | 318.582 | 1495.2 | 1539.2 | 1411.8 | 448.71 | 141.66 |
| | Mean | | | | | | | | |
| | CV % | 53.22 | 51.05 | 32.61 | 25.82 | 24.23 | 16.28 | 62.39 | 16.28 |
| | Geometric | | | | | | | | |
| | Mean | | | | | | | | |

Hepatic stiffness by transient elastography (TE) and magnetic resonance elastography (MRE), measured before and after dosing, changed by −11% (TE) or −6% (MRE) in the 50 mg, −7% (TE) or 4% (MRE) in the 150 mg, and −2% (TE) or 0% (MRE) in the 600 mg groups.

At the end of 4-week dosing, plasma levels of pro-C3, a liver fibrosis marker, were decreased from baseline by −8%, −1%, and −5% in the groups administered 50 mg, 150 mg, and 600 mg, respectively. At 2-week post-dose follow-up, pro-C3 levels were −7%, 8%, and 1% from baseline in the groups administered 50 mg, 150 mg, and 600 mg, respectively.

Overall improvement was also observed in insulin resistance by homeostatic model assessment for insulin resistance (HOMA-IR) after 4-week 25HC3S treatment. At the end of dosing, HOMA-IR was −22%, −18%, and 1% from baseline in the groups administered 50 mg, 150 mg, and 600 mg, respectively. At 2-week post-dose follow-up, it was −10% from baseline in the group administered 50 mg, and 17% and 3% in the groups administered 150 mg and 600 mg, respectively.

### Conclusions

This report presents pharmacokinetics of 25HC3S following oral administration to normal healthy and NASH subjects at the doses 50 mg (Cohort 1) to 200 mg (Cohort 2). Healthy subjects in Cohort 2 provided sufficient data to enable CL/F to be determined at 171.25±28.60 L/h and V_{z}/F at 434.05±128.07 L. This was supported by T_{1/2} results which remained relatively constant with mean values ranging from 1.674 hours to 2.511 hours across both healthy and NASH subject groups. AUC₀₋ₗₐₛₜ and AUC_{inf} were consistent within cohorts and tended, along with Cₘₐₓ, to increase in a less than proportional manner with increasing 25HC3S dose.

An 18-24% increase in Cₘₐₓ (geometric mean) was observed for NASH over healthy subjects. However the greater CV % geometric mean for NASH subjects made this observation inconclusive. Similarly when exposure (AUC parameters) was considered, potential increasing trends in NASH subjects (up to 31%) were countered by higher CV % geometric mean. Hence, it was concluded that no clear difference in pharmacokinetics occurred whether 25HC3S was administered to healthy subjects or NASH patients.

### EXAMPLE 3

### Objective

The objectives of this study were to determine the plasma pharmacokinetics of [4-¹⁴C]-25HC3S-derived radioactivity in male Sprague Dawley rats, determine the routes of elimination and excretion mass balance of [4-¹⁴C]-25HC3S-derived radioactivity in male Sprague Dawley rats, determine the tissue distribution and tissue pharmacokinetics of [4-¹⁴C]-25HC3S-derived radioactivity using quantitative whole body autoradiography methods in male Sprague Dawley and Long Evans rats following a single intravenous (bolus) dose, and to provide plasma, urine, and fecal homogenate samples for metabolite profiling of [4-¹⁴C]-25HC3S-derived radioactivity.

### Study Design

Nine male Sprague Dawley rats (Group 1) were designated for the pharmacokinetic phase, 3 male Sprague Dawley rats (Group 2) for the excretion mass balance phase, and 7 male Sprague Dawley rats (Group 3) and 9 male Long Evans rats (Group 4) for the tissue distribution phase. All animals received a single intravenous dose of [¹⁴C]-25HC3S at 10 mg/kg and a target radioactivity of 225 μCi/kg. Blood samples were collected from all Group 1 animals at approximately 0.083, 0.25, 0.5, 1, 2, 4, 8, 12, 24, 48, and 72 hours post-dose. Urine and feces were collected from all Group 2 animals periodically through 168 hours post-dose. At approximately 0.083, 0.5, 1, 4, 8, 24, and 168 hours post-dose for Group 3 and at approximately 0.083, 0.5, 1, 4, 8, 24, 168, 336, and 504 hours post-dose for Group 4, 1 animal/group/time point was anesthetized with isoflurane and a blood sample collected. Following blood collection, animals were euthanized by CO₂ inhalation and carcasses frozen in a dry ice/hexane bath for processing by quantitative whole body autoradiography. Whole blood, plasma, urine, feces, cage rinse, and cage wash were analyzed for total radioactivity by liquid scintillation counting.

### Results and Key Findings

After a single intravenous (bolus) dose of [4-¹⁴C]-25HC3S administered to rats at 10 mg/kg, the mean plasma C₀ was 25,900 ng-equiv./g, and AUCₗₐₛₜ was 27,900 h*ng-equiv./g. The terminal elimination phase T_{1/2} was 26.6 hours.

Based on the excretion data, approximately 100.2% of the dose administered was recovered over 168 hours in urine, feces, and cage rinse from rats following a single intravenous (bolus) dose of [4-¹⁴C]-25HC3S at 10 mg/kg. The majority of the recovered radioactivity was in feces (83.0%), indicating that biliary excretion is the primary route of excretion in rats.

After a single intravenous (bolus) dose of [4-¹⁴C]-25HC3S to male Sprague Dawley rats in Group 3 at 10 mg/kg, [4-¹⁴C]-25HC3S and/or its metabolites were broadly distributed and detected by quantitative whole body autoradiography in all tissues except the eye (lens). Plasma concentrations were similar to those determined in the pharmacokinetics phase. The whole blood Cₘₐₓ was 8530 ng-equiv/g, and AUCₗₐₛₜ was 25,200 h*ng-equiv./g. There was a negligible difference in plasma and whole blood exposure, as measured by the plasma:whole blood AUCₗₐₛₜ ratio of 0.79, indicating that the 25HC3S partitioned equally into plasma and blood cells. The T_{1/2} was 44.3 hours in plasma and 52.2 hours in whole blood; differences in plasma T_{1/2} between the PK phase and the QWBA phase are due to the difference in blood collection time points.

The Cₘₐₓ and AUCₗₐₛₜ for [4-¹⁴C]-25HC3S-derived radioactivity were highest in the liver: up to 87,900 ng-equiv./g and 364,000 hng/g, respectively. Kidney (all sections), small intestine (wall), lung, and adrenal gland concentrations ranged from 43,200 ng-equiv./g to 13,600 ng-equiv./g, higher than the maximum plasma concentration of 12,400 ng-equiv./g. Thymus, bone (femur), uveal tract, fat, testes, and brain concentrations were lowest relative to the other tissues: <5000 ng-equiv./g (around 1500 ng-equiv./g). Remaining tissues had concentrations between 5000 and 10,800 ng-equiv./g. The Tₘₐₓ was most often 0.083 to 0.5 hours post-dose. Concentrations were below quantitation limit in all tissues except adrenal gland, harderian gland, liver, and small intestine by 168 hours post-dose. As calculated using AUCiast, the tissue:plasma ratios were high for liver and small intestine (wall) at 11.4 and 7.44 , respectively. High liver and small intestine concentrations are consistent with extensive biliary (fecal) excretion following an intravenous dose. All other tissue:plasma ratios demonstrated limited affinity for remaining tissue types.

Administration of a single intravenous dose of [4-¹⁴C]-25HC3S to male Long Evans rats at 10 mg/kg revealed no substantial difference in plasma or whole blood concentrations over the first 168 hours postdose versus Sprague Dawley rats; plasma and whole blood concentrations were below quantitation limit in plasma and whole blood by 336 hours postdose in pigmented animals. There appeared to be no difference in binding to pigmented or non-pigmented skin or the uveal tract; for all tissues, the concentrations were below quantitation limit by 168 hours post-dose.

Plasma, urine, and feces from rats were analyzed for determination of 25HC3S related radiolabeled materials. Samples were profiled using high performance liquid chromatography with radiodetection and metabolic characterization was performed using mass spectrometry and tandem mass spectrometry analysis.

Plasma pools were made from Group 1 rats at the 0.083, 0.25, 0.5, and 1-hour collection time points. From these Group 1 sample pools and from a Group 3 0.083-hour plasma sample, the largest component present in the 0.083- and 0.25-hour collections was attributed to the parent 25HC3S representing about 58% to 92% of the radioactivity. Three metabolites present at >10% of the radioactivity in the 0.5- and 1-hour collections were M14 (up to 15% relative observed intensity), M24 (up to 13% relative observed intensity), and M28 (up to 83% relative observed intensity). Among the time points with suitable radioactivity for metabolite profiling and characterization (up to 1 hour postdose), approximately 54% of the exposure (AUC) to 25HC3S related radioactivity was attributable to 25HC3S, approximately 34% to M28, and the remainder to the minor metabolites.

Urine pools were prepared for Group 2 at 0 to 6 and 6 to 12 hours postdose. The largest component present was attributed to the parent 25HC3S representing about 78% to 93% of the radioactivity. A total of 4 metabolites were identified, although no metabolites were present at >1.2% of dose or >10% relative observed intensity. Four metabolites present at <10% relative observed intensity in at least 1 sample were M7 (<5% relative observed intensity), M16 (<3% relative observed intensity), M19 (<6% relative observed intensity), and M25 (<5% relative observed intensity).

Feces pools were prepared for Group 2 at 0 to 12, 12 to 24, and 24 to 48 hours postdose.

A total of fourteen metabolites were identified. Four metabolites present at ≥5% of dose were M1 (21% of dose and 23% to 30% relative observed intensity), M2 (7% of dose and 4% to 12% relative observed intensity), M3 (15% of dose and 13% to 23% relative observed intensity), and M4 (8% of dose and 6% to12% relative observed intensity). Parent 25HC3S was present at 2% of dose (1% to 5% relative observed intensity).

The primary metabolic pathways involved oxidation of 25HC3S resulting in the conversion of the sulfate group to a hydroxyl group followed by further oxidation to form bile acid structures related to deoxycholic acid and cholic acid or their isomers. In addition, glutathione conjugation of deoxycholic acid (or an isomer of deoxycholic acid) was suggested by the presence of a metabolite having the corresponding molecular weight for that structure. Neither desmosterol sulfate nor 25-hydroxycholesterol was detected in any of the plasma, urine, or feces samples.

### EXAMPLE 4

After a single oral (gavage) dose of [¹⁴C]-25HC3S administered to rats at 75 mg/kg, plasma Cₘₐₓ was 3800 ng equiv./g, and AUCiast was 96,400 h.ng equiv./g. The terminal elimination phase T_{1/2} was 27.3 hours.

Based on the excretion data, approximately 94.5% of the dose administered was recovered in urine, feces, and cage rinse from rats following a single oral (gavage) dose of [¹⁴C]-25HC3S at 75 mg/kg. The majority of the recovered radioactivity was in feces (94.2%), indicating that biliary excretion is the primary route of excretion for absorbed 25HC3S in rats.

After a single oral (gavage) dose of [¹⁴C]-25HC3S to male Sprague Dawley rats at 75 mg/kg, [¹⁴C]-25HC3S and/or its metabolites were broadly distributed and detected by quantitative whole body autoradiography in all tissues except the eye (lens). No [¹⁴C]-25HC3S-derived radioactivity was detected in the eye (lens). Plasma concentrations were similar to those determined in the pharmacokinetics phase, and were above the lower limit of quantitation. The whole blood Cₘₐₓ was 2850 ng equiv/g, and AUCₗₐₛₜ was 127,000 h.ng equiv./g. There was a negligible difference in plasma and whole blood exposure, as measured by the plasma:whole blood AUCₗₐₛₜ ratio of 1.12, indicating that the 25HC3S partitioned approximately equally into plasma and blood cells.

For the tissues analyzed by quantitative whole-body autoradiography, the Cₘₐₓ for [¹⁴C]-25HC3S-derived radioactivity, where measurable, was highest in the small intestine (wall) followed by the stomach (wall): 424,000 ng equiv./g and 204,000 ng equiv./g, respectively. Pancreas and liver concentrations ranged from 23,500 ng equiv./g to 28,100 ng equiv./g. Uveal tract and brain concentrations were lowest relative to the other tissues and were approximately 1000 ng equiv./g. Skin, thymus, prostate, and pituitary tissue concentrations were <3000 ng equiv./g. Remaining tissues had concentrations between 3600 ng-equiv./g and 10,700 ng equiv./g. The Tmax was 6 hours postdose or less. By 168 hours postdose, tissue concentrations were near or below the quantitation limit in all tissues except adrenal gland and liver. As calculated using AUClast, the tissue:plasma ratios were highest for the small intestine (wall, 15.4) followed by the liver and adrenal gland at 6.96 and 6.64, respectively. High liver and small intestine concentrations are consistent with oral administration and biliary (fecal) excretion. All other tissue:plasma ratios demonstrated limited affinity for remaining tissue types.

Radiolabeled components in plasma and feces extracts were profiled and identified using radio-high performance liquid chromatography (HPLC) and high performance liquid chromatography/mass spectrometry (HPLC/MS) methods.

There were no urine samples that contained sufficient radioactivity to require metabolite profiling and identification.

Plasma pools were prepared for Group 1 (75 mg/kg, [¹⁴C]-25HC3S) samples collected at 2, 4, and 6 hours post-dose. In the 2 hour postdose plasma, the primary radiolabeled component was parent 25HC3S which was present at 63% relative observed intensity (ROI) and a concentration of 2090 ng-equiv./g. One metabolite M29 was identified as 25-hydroxycholesterol with 37% ROI and a concentration of 1233 ng-equiv./g. The plasma collections at 4 and 6 hours post-dose did not contain sufficient concentrations for radioprofiling.

Feces pools were prepared for Group 2 (75 mg/kg, [¹⁴C]-25HC3S) samples collected from 0 to 24, 24 to 48, 48 to 72, 72 to 96, 96 to 120, 120 to 144, and 144 to 168 hours post-dose. A total of eleven metabolites were identified. None of the metabolites were present at ≥5% of dose. Metabolites present at 2-5% of dose were M1 (4.5% of total dose and 1%-69% ROI), M3 (4.6% of total dose and 1%-44% ROI), M4 (2.0% of total dose and 0%-10% ROI), M8 (3.1% of total dose and 1%-46% ROI), M29 (1.9% of total dose and 0%-2% ROI), and M30 (3.3% of total dose and 0%-5% ROI). The primary radiolabeled component was parent 25HC3S which was present at 71.1% of total dose (0%-88% ROI).

Radiolabeled desmosterol sulfate was not found in any of the plasma or feces samples.

The primary metabolic pathways involved oxidation of 25HC3S, resulting in the conversion of the sulfate group to a hydroxyl group followed by further oxidation to form bile acid structures related to deoxycholic acid and cholic acid or their isomers and 25-hydroxycholesterol.

1. A method of treating non-alcoholic steatohepatitis (NASH) in a human subject in need thereof, the method comprising orally administering to the subject 5-cholesten-3,25-diol, 3-sulfate (25HC3S) or salt thereof in an amount ranging from 1 mg/day to 100 mg/day.

2. A method of lowering serum alanine aminotransferase (ALT) levels in a human subject having non-alcoholic steatohepatitis (NASH), comprising:
orally administering to the subject 5-cholesten-3,25-diol, 3-sulfate (25HC3S) or salt thereof in an amount ranging from 1 mg/day to 100 mg/day.

3. A method of lowering liver stiffness in a human subject having non-alcoholic steatohepatitis (NASH), comprising:
orally administering to the subject 5-cholesten-3,25-diol, 3-sulfate (25HC3S) or salt thereof in an amount ranging from 1 mg/day to 100 mg/day.

4. A method of lowering serum triglycerides in a human subject having non-alcoholic steatohepatitis (NASH), comprising:
orally administering to the subject 5-cholesten-3,25-diol, 3-sulfate (25HC3S) or salt thereof in an amount ranging from 1 mg/day to 100 mg/day.

5. A method of lowering serum triglycerides in a human subject having non-alcoholic steatohepatitis (NASH) and having triglycerides ≥200 mg/dL prior to treatment, comprising:
orally administering to the subject 5-cholesten-3,25-diol, 3-sulfate (25HC3S) or salt thereof in an amount ranging from 1 mg/day to 100 mg/day.

6. The method according to any one of claims 1 to 5, wherein the orally administering comprises orally administering the 25HC3S or salt thereof ranging from about 10 mg/day to about 80 mg/day.

7. The method according to any one of claims 1 to 5, wherein the orally administering comprises orally administering the 25HC3S or salt thereof ranging from about 30 mg/day to about 70 mg/day.

8. The method of any one of claims 1 to 7, wherein a total amount per kg of 25HC3S or salt thereof that is orally administered to the subject ranges from about 0.1 mg/kg/day to about 5 mg/kg/day.

9. The method of claim 8, wherein the total amount per kg ranges from about 0.2 mg/kg/day to about 4 mg/kg/day.

10. The method of claim 8, wherein the total amount per kg ranges from about 0.3 mg/kg/day to about 3 mg/kg/day.

11. The method of claim 8, wherein the total amount per kg ranges from about 0.4 mg/kg/day to about 2 mg/kg/day.

12. The method of any one of claims 1 to 11, wherein the orally administering comprises orally administering a plurality of doses of the 25HC3S or salt thereof.

13. The method of claim 12, wherein the doses are orally administered at a frequency ranging from once weekly to three times a day.

14. The method of claim 13, wherein the doses are orally administered once a day.

15. The method of claim 13, wherein the doses are orally administered twice a day.

16. The method of any one of claims 12 to 15, wherein the orally administering comprises orally administering for a dosing period of at least 7 days, such as at least 14 days, at least 28 days, at least 3 months, at least 6 months, or at least 1 year.

17. The method of any one of claims 1 to 16, wherein the 25HC3S or salt thereof is orally administered in a formulation comprising the 25HC3S or salt thereof and a pharmaceutically acceptable carrier.

18. The method of any one of claims 1 to 17, wherein the 25HC3S or salt thereof comprises a salt of 25HC3S.

19. The method of claim 18, wherein the salt of 25HC3S is sodium salt.

20. The method of any one of claims 1 to 19, wherein the human subject has a magnetic resonance imaging-proton density fat fraction (MRI-PDFF) prior to treatment of at least 5%.

21. The method of any one of claims 1 to 20, wherein the human subject has a magnetic resonance elastography (MRE) prior to treatment ≥2.75 kPa.

22. The method of any one of claims 1 to 21, wherein the subject exhibits a half-life time of 25HC3S in the plasma after administration (T_{1/2}) ranging from about 1 hour to about 5 hours or from about 1.5 hour to about 4 hours.

23. The method of any one of claims 1 to 22, wherein the subject exhibits a Cmax of 25HC3S ranging from about 25 ng/mL to about 200 ng/mL, from about 50 ng/mL to about 150 ng/mL, or from about 75 ng/mL to about 125 ng/mL.

24. The method of any one of claims 1 to 23, wherein the subject exhibits a Cmax of 25HC3S ranging from about 100 ng/mL to about 300 ng/mL, from about 120 ng/mL to about 250 ng/mL, or from about 150 ng/mL to about 200 ng/mL, per 100 mg of orally administered 25HC3S or salt thereof.

25. The method of any one of claims 1 to 24, wherein the subject exhibits an AUCinf of 25HC3S ranging from about 300 ng*h/mL to about 1000 ng*h/mL, about 400 ng*h/mL to about 900 ng*h/mL, or from about 500 ng*h/mL to about 800 ng*h/mL.

26. The method of any one of claims 1 to 25, wherein the subject exhibits an AUCinf of 25HC3S ranging from about 600 ng*h/mL to about 1000 ng*h/mL, about 700 ng*h/mL to about 900 ng*h/mL, or from about 800 ng*h/mL to about 900 ng*h/mL, per 100 mg of orally administered 25HC3S or salt thereof.

27. The method of any one of claims 1 to 29, wherein the subject exhibits an apparent volume of distribution (Vz/F) of 25HC3S ranging from about 300 L to about 1000 L, about 400 L to about 900 L, or from about 500 L to about 800 L.

28. The method of any one of claims 1 to 27, wherein the subject exhibits an apparent clearance (CL/F) of 25HC3S ranging from about 100 L to about 200 L/h, about 110 L/h to about 180 L/h, or from about 120 L/h to about 160 L/h.

29. The method of any one of claims 1 to 28, wherein the subject is taking a lipid lowering drug, such as at least one of a statin, fenofibrate, omega-3 fatty acid, icosapent ethyl, and fish oil, or further comprising administering a lipid lowering drug, such as at least one of a statin, fenofibrate, omega-3 fatty acid, icosapent ethyl, and fish oil, to the subject.

30. The method of any one of claims 1 to 29, wherein the subject is taking at least one of atorvastatin, fluvastatin, lovastatin, pitavastatin, pravastatin, rosuvastatin, and simvastatin, or further comprising administering to the subject at least one of atorvastatin, fluvastatin, lovastatin, pitavastatin, pravastatin, rosuvastatin, and simvastatin.

31. 5-cholesten-3,25-diol, 3-sulfate (25HC3S) or salt thereof for use in a method of treating non-alcoholic steatohepatitis (NASH) in a human subject in need thereof, wherein the method is as defined in any one of claims 1 to 30.

32. 5-cholesten-3,25-diol, 3-sulfate (25HC3S) or salt thereof for use in a method of treating non-alcoholic steatohepatitis (NASH) in a human subject in need thereof, the human subject having triglycerides ≥200 mg/dL prior to treatment, wherein the method is as defined in any one of claims 1 to 30.

33. Use of 5-cholesten-3,25-diol, 3-sulfate (25HC3S) or salt thereof in a method for the manufacture of a medicament for use in a method of treating non-alcoholic steatohepatitis (NASH) in a human subject in need thereof, wherein the method is as defined in any one of claims 1 to 30.

34. Use of 5-cholesten-3,25-diol, 3-sulfate (25HC3S) or salt thereof in a method for the manufacture of a medicament for use in a method of treating non-alcoholic steatohepatitis (NASH) in a human subject in need thereof, the human subject having triglycerides ≥200 mg/dL prior to treatment, wherein the method is as defined in any one of claims 1 to 30.

35. 5-cholesten-3,25-diol, 3-sulfate (25HC3S) or salt thereof for use in a method of treating non-alcoholic steatohepatitis (NASH) in a human subject in need thereof, wherein said human subject is receiving statin therapy.

36. 5-cholesten-3,25-diol, 3-sulfate (25HC3S) or salt thereof for use according to claim 35, wherein said statin therapy comprises administration of at least one of atorvastatin, fluvastatin, lovastatin, pitavastatin, pravastatin, rosuvastatin, and simvastatin.

37. 5-cholesten-3,25-diol, 3-sulfate (25HC3S) or salt thereof for use according to claim 35 or 36, wherein said method is a method as defined in any one of claims 1-30, and optionally wherein the human subject has triglycerides ≥200 mg/dL prior to treatment.

38. 5-cholesten-3,25-diol, 3-sulfate (25HC3S) or salt thereof for use in a method of treating non-alcoholic steatohepatitis (NASH) in a human subject in need thereof by co-administration with at least one statin, optionally wherein said at least one statin comprises at least one of atorvastatin, fluvastatin, lovastatin, pitavastatin, pravastatin, rosuvastatin, and simvastatin.

39. 5-cholesten-3,25-diol, 3-sulfate (25HC3S) or salt thereof for use according to claim 38, wherein said human subject is one receiving statin therapy prior to commencing said method, and optionally wherein said statin therapy comprises administration of the same statin or statins that is or are co-administered with said 25HC3S or salt thereof in said method.

40. 5-cholesten-3,25-diol, 3-sulfate (25HC3S) or salt thereof for use according to claim 38 or 39, wherein said method is a method as defined in any one of claims 1-30, and optionally wherein the human subject has triglycerides ≥200 mg/dL prior to treatment.

41. Use of 5-cholesten-3,25-diol, 3-sulfate (25HC3S) or salt thereof in a method for the manufacture of a medicament for use in a method of treating non-alcoholic steatohepatitis (NASH) in a human subject in need thereof, wherein said human subject is receiving statin therapy.

42. Use according to claim 41, wherein said statin therapy comprises administration of at least one of atorvastatin, fluvastatin, lovastatin, pitavastatin, pravastatin, rosuvastatin, and simvastatin.

43. Use according to claim 41 or 42, wherein said method of treating is a method as defined in any one of claims 1-30, and optionally wherein the human subject has triglycerides ≥200 mg/dL prior to treatment.

44. Use of 5-cholesten-3,25-diol, 3-sulfate (25HC3S) or salt thereof in a method for the manufacture of a medicament for use in a method of treating non-alcoholic steatohepatitis (NASH) in a human subject in need thereof by co-administration with at least one statin, optionally wherein said at least one statin comprises at least one of atorvastatin, fluvastatin, lovastatin, pitavastatin, pravastatin, rosuvastatin, and simvastatin.

45. Use according to claim 44, wherein said human subject is one receiving statin therapy prior to commencing said method, and optionally wherein said statin therapy comprises administration of the same statin or statins that is or are co-administered with said 25HC3S or salt thereof in said method.

46. Use according to claim 44 or 45, wherein said method of treating is a method as defined in any one of claims 1-30, and optionally wherein the human subject has triglycerides ≥200 mg/dL prior to treatment.
